(19) United States
(12) Patent Application Publication
(10) Pub. No.: US 2005/0090868 A1
(43) Pub. Date: Apr. 28, 2005

(54) Defibrillation signal, method and device for adjusting defibrillation energy relative to a patient's transthoracic resistance

(76) Inventor: Albert Cansell, Wissembourg (FR)

Correspondence Address: SHOEMAKER AND MATTARE, LTD, 10 POST OFFICE ROAD - SUITE 110, SILVER SPRING, MD, 20910, US

(21) Appl. No.: 10/499,650

(22) PCT Filed: Dec. 23, 2002
(86) PCT No.: PCT/EP02/14723

(30) Foreign Application Priority Data
Dec. 28, 2001 (FR) 01/17019

(51) Int. Cl. A61N 1/39

(52) U.S. Cl. 607/5

(57) ABSTRACT

The invention concerns a method which consists in measuring the patient's transthoracic resistance between two defibrillation electrodes applied to the patient during a defibrillation impact by using the first elementary pulse derived from chopping and modulation of the defibrillation pulse(s) to measure the patient's transthoracic resistance, the following elementary pulses being modulated so as to deliver to the patient the pre-selected or intended energy, independently of his transthoracic resistance between the defibrillation electrodes and without increasing the total duration of the series of impulses. The device consists of a user interface (1), a microcontroller (2), a high voltage generator (3), a high voltage circuit (4) and a wave form generator (5). The present invention is of interest to manufacturers of defibrillators.

### FIELD OF THE INVENTION

The present invention pertains to the field of medicine and more particularly concerns cardiac defibrillation and cardioversion in external or transthoracic application, and it relates to a method for automatically compensating and adjusting the defibrillation energy so as to deliver all the selected energy to the patient, irrespective of the patient's resistance between the electrodes that are applied to him or her.

### BACKGROUND OF THE INVENTION

There are currently defibrillators with biphasic waves and chopped biphasic waves, that is to say ones that are truncate in order to form a series of elementary pulses at a high frequency (see, for example: A. Cansell, La Revue des SAMU, 2000, 20.280-284).

One possible method that allows the patient's resistance to be taken into account consists in measuring this resistance by a current with low intensity and high frequency before the defibrillation shock is applied, and in automatically adjusting the energy for a preselected current (see, for example: Kerber et al. Circulation 1984, 70, 303-308; Dalzell et al. Brit Heart J 1989, 61, 502-505). This method has the drawback of providing a measurement of the resistance with a limited precision (of the order of ñ 10-15 ohms) and of taking a time of the order of a few seconds, which is added to the charging time of the capacitor for delivering the shock.

Another method consists in measuring the start of the decreasing slope of an untruncated defibrillation pulse, on the basis of which edge the patient's resistance is calculated in view of extending the duration of this pulse until the preselected energy has been delivered (see U.S. Pat. No. 5,593,427). In patients who have a high resistance, the energy thus continues to be applied at pulse durations in excess of 5 to 6 ms and even up to 10 ms for the first phase, which is physiologically unsatisfactory. Beyond a duration of about 5 ms, the delivered energy is ineffective and sometimes harmful, and it may cause refibrillation in certain cases. In the case of biphasic pulses, furthermore, the distribution of the energies in phase I and phase 2 differs for varying patient resistances. The effect of this is that the ratio between the two phases will be modified by this and may not therefore have its optimum value (cf. for example: A. Cansell, La Revue des SAMU, 2000, 20.280-284).

A variant of this method avoids the latter drawback and permits an optimum ratio between the amplitudes of the two phases, while limiting their durations to a maximum of 4 to 5 ms per phase and by applying the measurement and the compensation to each of the two phases (see Krasteva et al. J. Med. Eng. Technol. 24, 210-214). In this way, the transmembrane potential is raised above the defibrillation threshold in less than 4 to 5 ms and returns to its initial level toward the end of the phase 2 pulse. The deficiency of this solution is the difficulty in maintaining adequate compensation with such short pulses (4 to 5 ms) for patient resistances lying in the range of from 100 to 150 ohms, bearing in mind that values of up to about 180 ohms are possible.

A third method consists in applying a low-voltage pulse followed by measurement of the corresponding current, thereby obtaining the resistance on the basis of which a preselected current is imposed through the patient's thorax (See: Mittal et al. J. Am. College of Cardiology, 1999, 34, 1595-1601). Measuring the current with a voltage below the defibrillation threshold can give different values of patient resistance, which moreover are generally higher than those obtained with the voltage that will actually be applied. Furthermore, this solution does not allow for the optimum ratio of the amplitudes or energies of the two phases, which is not obtained satisfactorily. The second phase does no more than discharge the energy resulting from phase 1 to the patient, so that the amplitude of the second phase is dictated by this residual energy. It is an object of the present invention to overcome some of the drawbacks and limitations mentioned above.

### SUMMARY OF THE INVENTION

The inventors have already created, and demonstrated the effectiveness of, a category of biphasic pulses that are truncated or chopped into elementary pulses modulated by frequency or duration, or both, which make it possible to obtain averaged energy or current waves with different forms and shapes, as described in the patent application published in France under the No. 2808694.

The inventors have found that after the patient's resistance has been measured by using the first actual elementary pulse applied to the patient, the subsequent elementary pulses can be modulated so as to deliver the preselected energy to the patient, and to do so independently of the transthoracic resistance, without having to use total durations of more than 4 to 5 ms for each phase. Since this duration is optimum for maximal efficacy, such compensation turns out to be important. This is because if the patient's resistance increases as a voltage is applied to the patient, the current flowing through the patient will drop proportionally, and the energy applied to the patient will drop significantly. The principle proposed by the present invention has the advantage of achieving a substantially constant delivered energy corresponding to the reference value, given at 50 ohms, desired by the operator, without extending the total duration of the defibrillation pulse, which in most common practice is the envelope of the pulse train. The invention therefore relates to a method for generating a signal of elementary pulses, a device for generating such a signal and to such a signal per se.

One practical way of providing compensation of the applied energy throughout the range of patient resistances, extending for example between 40 and 180 O, consists in segmenting this range into a certain number of resistance bands and of associating a modulation with each resistance band, according to a preestablished law (also referred to below as a switching, chopping or modulation law) intended to compensate and adjust the value of the energy as a function of a given patient's resistance.

### BRIEF DESCRIPTION OF THE DRAWINGS

The invention will be understood more clearly from the following description, which refers to preferred embodiments that are given by way of nonlimiting examples and are explained with reference to the appended schematic drawings, in which:

FIG. 1 is an overall functional block diagram illustrating the basic principle of the invention,

FIG. 2 is the detailed diagram of the high-voltage circuit of the generator device according to the invention,

FIG. 3, are graphs representing some examples of 4, 5, 6 truncated or chopped and modulated defibrillation pulses, each corresponding to different patient resistances, and

FIG. 7 is a comparative graph showing the relative energy loss in a percentage as a function of the patient's resistance, with and without the compensation resulting from the invention.

### DESCRIPTION OF THE PREFERRED EMBODIMENTS

The present invention is based on the general inventive concept according to which a method for automatically compensating and adjusting the defibrillation or cardioversion energy with a view to the delivery, by means of defibrillation electrodes connected to a defibrillator, of at least one defibrillation pulse with the selected total energy value in the form of at least one defibrillation pulse or phase of given polarity consisting of at least one series of elementary pulses is characterized in that the patient's transthoracic resistance is measured between the two electrodes applied to the patient by using the first elementary pulse of the defibrillation pulse, and in that the following pulses and the consecutive pauses are modulated according to a modulation law so as to deliver the preselected or intended energy to the patient, and to do so irrespective of his or her transthoracic resistance measured between the defibrillation electrodes and while keeping substantially constant the total duration of the envelope of the series of elementary defibrillation pulses corresponding to at least one phase of given polarity.

This method is also characterized in that the spread of the known resistances of patients is divided into a plurality of ranges, and in that a distinct modulation law corresponds to each range.

This method is also characterized in that a phase of given polarity has a predetermined duration.

This method is also characterized in that the determined duration of each phase of given polarity is from 4 to 5 ms.

This method is furthermore characterized in that the defibrillation pulse is a biphasic defibrillation pulse with two phases of different given polarity which is truncated into elementary pulses, the first of which elementary pulses is used for measuring the patient's transthoracic resistance with a view to modulating the following ones in order to deliver a predetermined defibrillation energy.

In the case of operation with a biphasic wave, the free dimensioning of the amplitude of the second phase by means of a second capacitor, which is charged to the desired voltage, is a way of obtaining a given proportion between the amplitude, in particular in terms of current, electric charge (intensity multiplied by time) or energy of the second phase with respect to the first phase, if the durations of the two phases are meant to remain constant. If only a single capacitor is used, however, the amplitude of the second phase will be equal to the amplitude of the first phase, which is inverted, and the proportions of the current, the electric charge (intensity multiplied by time) or energy of the two phases will not be freely selectable.

There is nevertheless a way of avoiding the use of two capacitors while still being able to obtain a predetermined proportion of the second phase with respect to the first phase, this proportion being expressed either as units of electric charge or as energy of the two phases.

This method consists in using a single capacitor, in appropriately dosing the total charge or the total energy needed for the second phase by controlling the durations either of the individual pulses of the second phase or by controlling those of the pauses of this second phase, or by controlling both at the same time, without modifying the total duration of the second phase. It is thus possible to discharge a predetermined energy in a given time.

According to one embodiment, the selected switching and modulation law may consist, for example—for a given law—of a fixed frequency and a duty ratio (ratio between the duration of the pulse and the duration of the cycle) that is constant (for a given truncation and modulation law). Depending on the energy content to be obtained in the two phases, this constant duty ratio—for a given modulation law—may take any values between 0% and 100%.

If, for technical or other reasons, it is expedient that the duty constant cycle selected for a given modulation law should be the same for both phases, it will not normally be possible for the electric charge (or energy) content contained in the second phase to be controlled freely and independently so that the latter has a desired proportion with respect to the electric charge (or energy) content of the first phase, if it is furthermore necessary to comply with the initial conditions of having a duration of the second phase that is constant and equal to the first, from 4 to 5 ms.

With the aim of finding a solution to this problem, more thorough recent experiments and studies have been carried out by the inventors regarding the physiologically optimum durations and the configuration (truncated or not) of the two phases. These studies have led to the following findings. They have confirmed that, on the one hand as regards the first phase, in order to have the highest possible efficacy, this ought to be truncated according to the principles explained above and have a duration of from 4 to 5 ms, as indicated. The reason for this finding is due to the fact that, physiologically speaking, it is the first phase which is essentially responsible for the defibrillation effect. This is the active part of the defibrillation, for which the truncation plays a very important role together with the duration of the phase, which is conditioned by the excitation time of the cells. On the other hand as regards the second phase, however, this is not involved directly in the act of defibrillation, but only has the purpose of eliminating the surplus electrical charges accumulated in the heart as a result of the first phase. The effect of eliminating these charges is that refibrillation phenomena are avoided, which makes it possible to retain the defibrillation effect obtained by the 1 st phase. This fact, already known in the case of untruncated traditional biphasic waves, has been confirmed by the inventors when a truncated first phase is used. The inventors then put forward the hypothesis that, owing to this rather passive role of the 2nd phase, perhaps the truncation and the duration of this phase play no role. This hypothesis has been confirmed by the inventors' studies: they found that after a first phase which is truncated and properly dimensioned for defibrillation, the second phase could equally well be truncated and have a duration whose value may be variable, without this having any influence on the efficacy, so long as its charge (or energy) content has an appropriate proportion with respect to the charge (or energy) content of the 1 st phase, or be not truncated while having a duration whose value may also be variable, without this having any influence on the efficacy, so long as its charge (or energy) content has an appropriate proportion with respect to the charge (or energy) content of the 1 st phase.

According to another embodiment of the invention, as regards the resistance measurement by means of the first pulse and the consecutive selection of a switching and modulation law, the inventors have found that it might be advantageous if the measurement of the patient's resistance and the selection of the truncation and modulation law could actually be carried out during the first pulse. In this way, the first cycle (pulse-pause) could already use the selected modulation law and form an integral part of it, for example so that the first cycle may even have a duty ratio identical to another in the series, or it may even be identical to all the cycles if the duty ratio has been selected to be constant, as in the example given above.

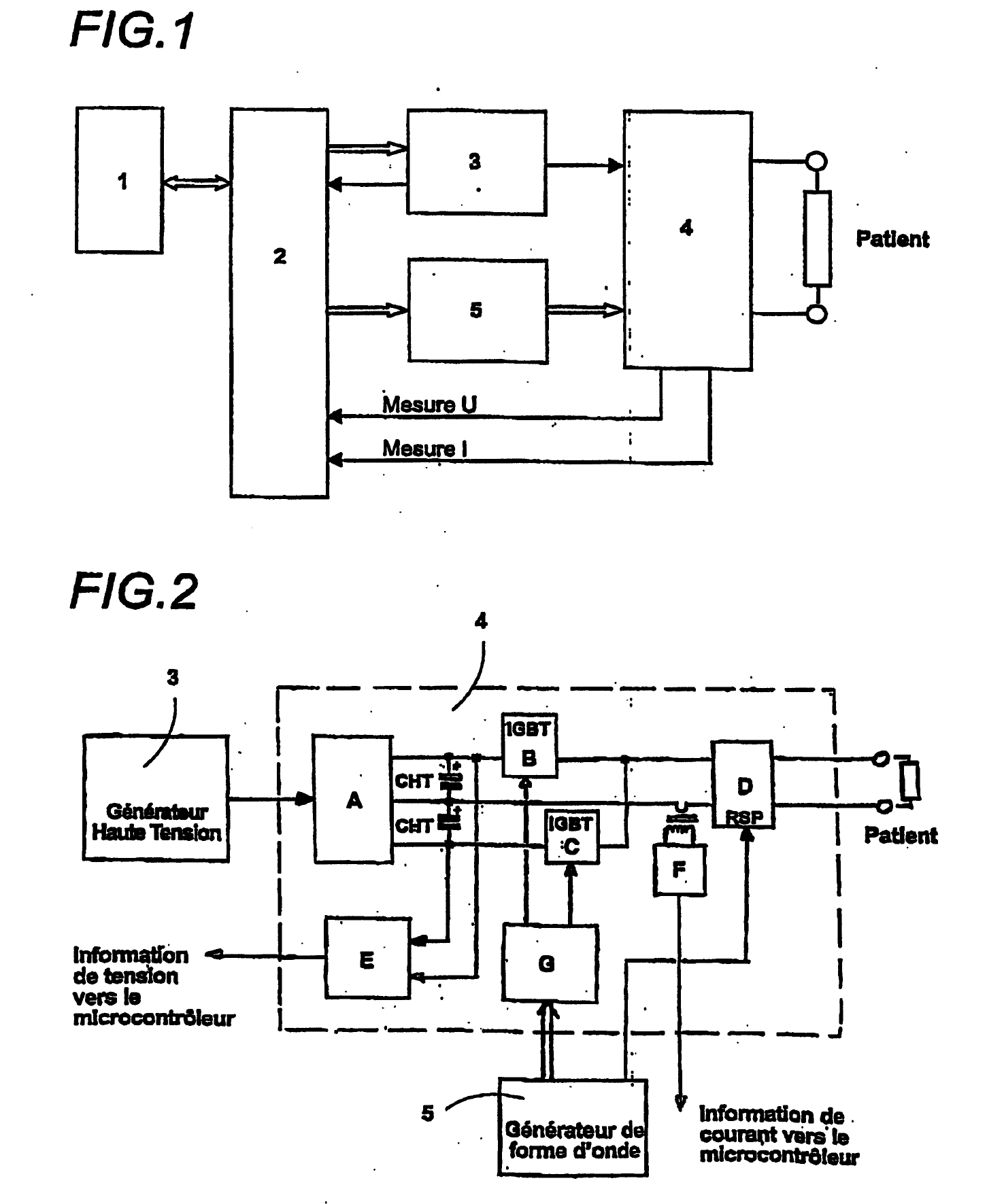

Referring to FIG. 1, the basic diagram illustrating the basic principle of the invention is composed of the following elements.

The diagram in FIG. 1 shows a user interface 1, a microcontroller 2, a high-voltage generator 3, a high-voltage circuit 4, and a waveform generator 5 which will be described below.

The high-voltage generator 4 is connected to the microcontroller 2 by a voltage measurement link and by an intensity measurement link, which are respectively denoted by measurement U and measurement I.

The user interface 1 is a subassembly which ensures the interfacing between the external control instruments and the defibrillation unit.

The microcontroller 2 is a functional unit which carries out all of the operations needed in order to deliver a defibrillation shock, when such is required. It generates all of the control signals needed for charging and discharging the high-voltage capacitor(s) HVC by means of the subassemblies generating high voltage 3, high-voltage circuit 4 and waveform generator 5.

The microcontroller 2 operates the high-voltage generator 3 in order to charge one or more high-voltage capacitor(s) HVC to the preselected energy value. When the preselected energy is reached, the microcontroller 2 enables discharging of the high-voltage capacitor(s) HVC. During the shock, the microcontroller 2 operates the patient safety relay denoted by PSR, as well as the waveform generator 5 which controls the high-voltage IGBT switches that generate the defibrillation wave. The microcontroller 2 determines the value of the resistance presented by the patient during the shock, on the basis of the current and voltage information measurements given by the first elementary pulse, in order to compensate and adjust the energy of the defibrillation wave, which is for example a biphasic defibrillation wave, as a function of the patient's resistance.

The high-voltage generator 3 is operated by the microcontroller 2. It transfers the energy from the supply source to the high-voltage capacitor(s) HVC until the preselected value of the energy is reached.

The high-voltage circuit 4 discharges the high-voltage capacitor(s) HVC via the switching circuits with IGBT switches and the patient relay denoted by PSR. The high-voltage circuit 4 is operated by the following two subassemblies: the microcontroller 2 and the waveform generator 5. In order to discharge the high-voltage capacitor(s) HVC for safety by default, the high-voltage circuit 4 also comprises a safety discharge circuit. The high-voltage circuit 4 furthermore measures the charging voltage of the high-voltage capacitor(s) HVC, and also measures the current flowing through the patient during the defibrillation shock.

The waveform generator 5 controls the switching circuits with IGBT switches in order to generate a pulsed biphasic wave. The waveform generator 5 is operated by the microcontroller 2 in order to compensate the pulsed biphasic wave as a function of the patient's resistance.

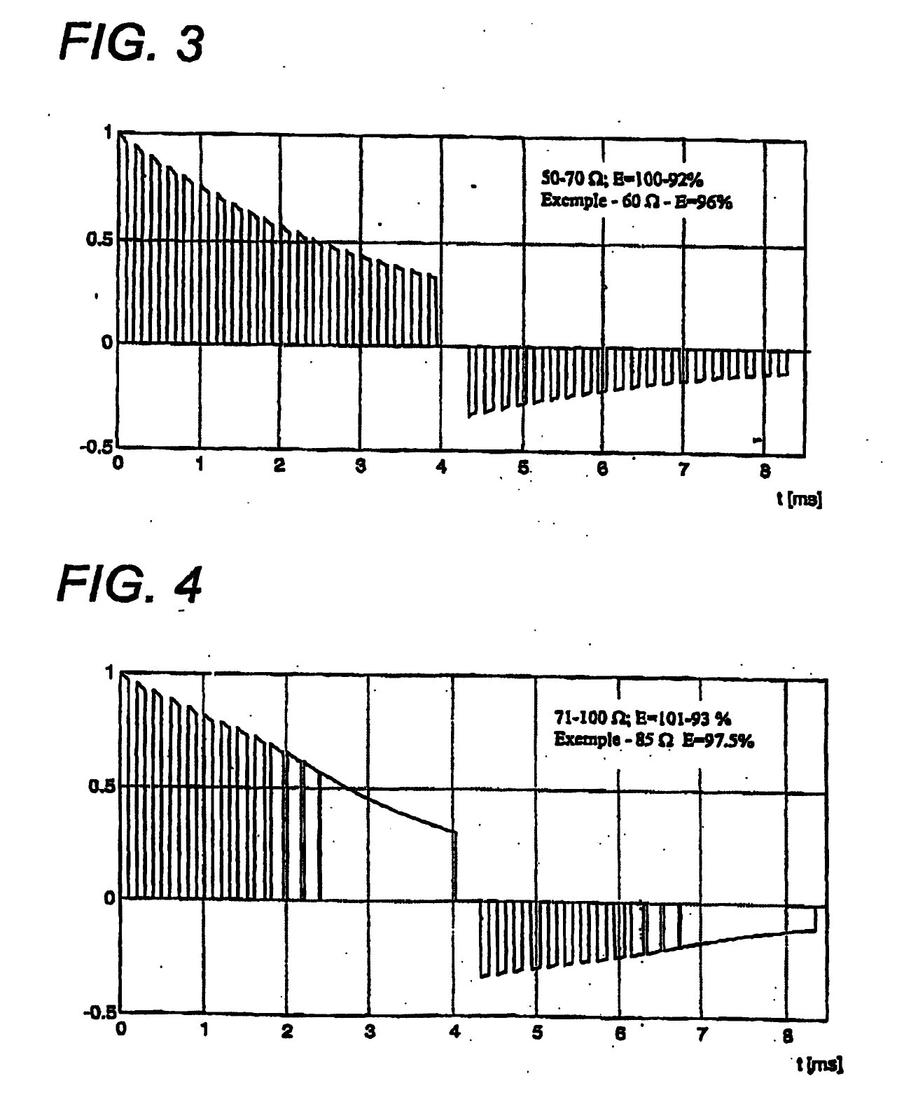

Details of the high-voltage circuit 4 will be described below by way of a nonlimiting example, with reference to its functional blocks which are represented in the diagram of FIG. 2.

It is composed of the blocks denoted by A, B, C, D, E and F in FIG. 2.

A. High-voltage transformer and rectifier
B. Semiconductor switch
C. Semiconductor switch
D. Electromechanical switch
E. Conditioning of the voltage information
F. Conditioning of the current information
G. Conditioning of the IGBT control information

The role of each of these functional modules will be indicated below.

The high-voltage transformer and rectifier A transfers energy between the supply source and the two high-voltage capacitors HVC, which may be replaced by a single capacitor, by means of the high voltage generator 3. Owing to its configuration, the high-voltage transformer and rectifier subassembly makes it possible to charge the high-voltage capacitor HVC, or the two high-voltage capacitors HVC, simultaneously to the value of the energy selected beforehand.

The two semiconductor switching subassemblies B and C generate the biphasic defibrillation wave with elementary pulses used for the shock, by means of the operating signals delivered by the waveform generator subassembly 5. For example these two subassemblies use semiconductor switches known by the abbreviation IGBT in order to fulfill this function. Each subassembly B or C may consist of a plurality of IGBT switches in combination.

The electromechanical switch subassembly D allows the patient to be DC isolated by reducing the leakage currents internal to the IGBT semiconductor switches that are used here. This electromechanical switch subassembly D is a relay, which is operated by the waveform generator 5 when the defibrillation shock is being applied. It is referred to as the patient safety relay PSR in this description.

The subassembly E for conditioning the voltage information carries out the measurement of the charging voltage, denoted by measurement U in FIG. 1, corresponding to the single high-voltage capacitor HVC, or to the two high-voltage capacitors HVC, so that this can be utilized by the microcontroller 2 in order to determine the patient's resistance.

The subassembly F for conditioning the current information carries out the isolated measurement of the defibrillation current at the start of the defibrillation shock, which is indicated by measurement of I in FIG. 1. Utilization of the two items of information about voltage and current allows the microcontroller 2 to determine the patient's resistance during the defibrillation shock, in order to adapt the form factor and, more generally, the pulse modulation of the defibrillation wave, which is for example a biphasic defibrillation wave.

The subassembly G for conditioning the control information carries out the interfacing and DC isolation needed for the operating signals generated by the waveform generator 5, in order to control the semiconductor switches B and C. The signals delivered by this subassembly G turn on and actively turn off the two IGBT semiconductor subassemblies in order to generate the defibrillation waveform suited to the patient's resistance during the defibrillation.

Of course, the way in which the functional blocks are combined as described above is only one example among others that could fulfill the same general function.

In the case of a single capacitor, for instance, a bridge structure for discharging it in two different polarity phases, or any other circuit layout or arrangement, lies fully within the scope of the present invention.

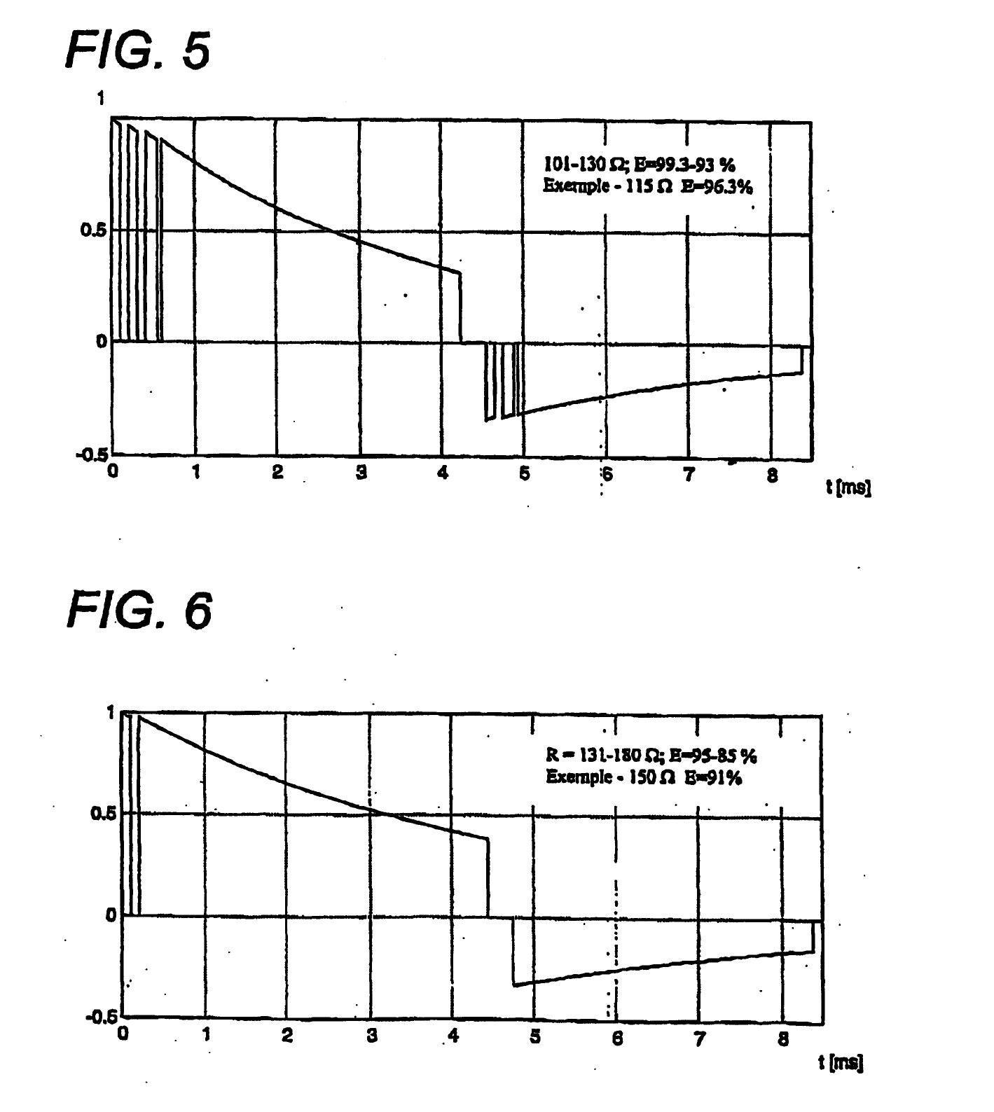

The curves of the examples represented in FIGS. 3 to 6 represent some examples of defibrillation pulses truncated or chopped into elementary pulses, each corresponding to different resistances, which are grouped as a plurality of subsets for a succession of value ranges. The first elementary pulse is used for measuring the transthoracic resistance, as explained above. The subsequent elementary pulses are modulated so as to ensure application of the preselected energy for various patient resistances.

FIG. 3 gives the shape of the defibrillation pulse corresponding to the band of from 40 to 70 ohms. The curve that is represented corresponds more particularly to a patient resistance of 60 ohms. In this domain of resistance values close to the reference value of 50 ohms, it may be noted that the form factor of the defibrillation pulses is constant and equal to 1, that is to say it is not modulated. The total energy content of the elementary pulse trains of the two phases is therefore the selected or intended reference value.

FIG. 4 gives the shape of the defibrillation pulse corresponding to the band of from 71 to 100 ohms. The curve that is represented corresponds more particularly to a patient resistance of 85 ohms. It may be noted that the form factor of the pulses is now variable in this domain of resistances, that is to say it is modulated. In particular, it can be seen that the last elementary pulse of the first phase is very broad, for example. The one before it is less broad, and-the pause between the two is very short. This is repeated as far as the first pulse, which has an identical width to the first elementary pulse in FIG. 3.

FIGS. 5 and 6 respectively correspond to resistance bands of from 101 to 130 ohms and from 131 to 180 ohms, showing a progressively larger filling effect for the envelope of the phase.

It may be noted in FIGS. 3 to 6 that the duration of each phase is substantially constant, and lies in an interval extending up to approximately 4 to 5 ms. As the resistance increases, the envelopes of the phases are filled up starting from the end of the phases. It will therefore be understood that the energy content also increases, which compensates for the loss due to the reduction in the current consecutive to the increase in resistance.

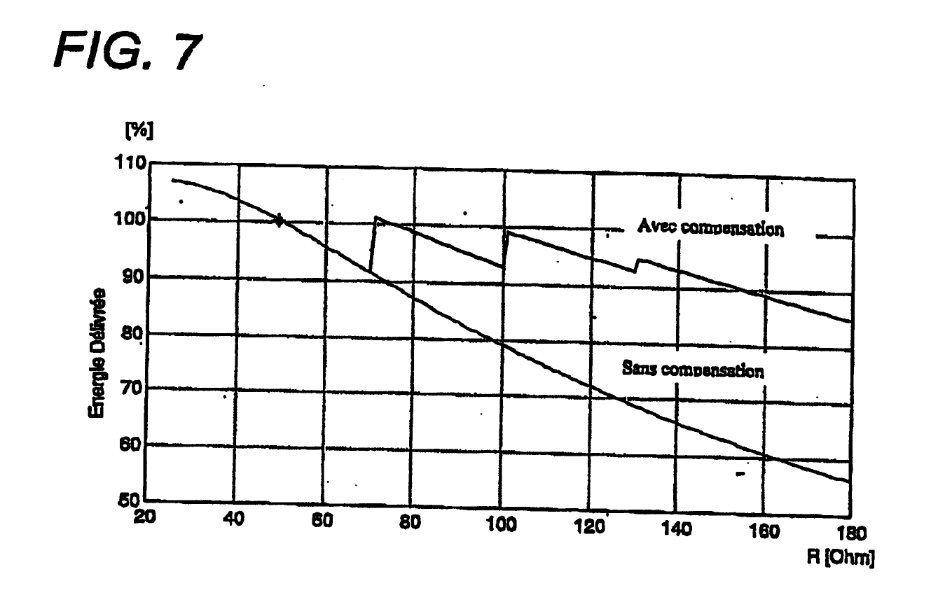

FIG. 7 shows the energy loss in a percentage as a function of the patient's resistance. The point (*) indicates the position on the curve corresponding to 100% of the energy for 50 ohms, which is the usual reference stipulated by the standards for indicating the energy.

The solid curve is the energy loss without compensation. The broken curve shows the compensation for energy by changing the modulation. In the example which is given, we have provided the following four bands: from 40 to 70 O: without compensation; from 70 to 100 O —from 100 O to 130 O—from 130 to 180 O: with compensation. The range of resistances may of course include a larger number of bands which are narrower, for example 10 ohm bands: from 50 to 60 ohms, from 60 to 70 ohms, etc.

The invention is not limited to a monophasic defibrillation pulse shape, but rather covers all the ones which are known and used at present, in particular biphasic defibrillation pulses.

This principle of compensating and adjusting the energy as a function of the patient's resistance is suitable for all the applications of defibrillation. Whether this may be ventricular defibrillation (in intensive care, in an electrophysiology laboratory or in prehospital reanimation), or in implantable defibrillation or in cardioversion (atrial defibrillation).

1. A defibrillation or cardioversion signal in view of the delivery, by means of defibrillation electrodes connected to a defibrillator, of at least one defibrillation pulse consisting of at least one series of elementary pulses, characterized in that it is formed by a first elementary pulse which is distinct from the others and is used for measuring the patient's transthoracic resistance between the electrodes applied to the patient, and in that the following pulses and the consecutive pauses are modulated on the basis of the patient's measured transthoracic resistance according to a modulation law so as to deliver the preselected or intended energy to the patient, and to do so irrespective of his or her transthoracic resistance measured between the defibrillation electrodes and while keeping substantially constant the total duration of the envelope of the series of elementary defibrillation pulses corresponding to at least one phase of given polarity.

2. The defibrillation signal as claimed in claim 1, characterized in that the defibrillation pulse is biphasic and includes two different phases of different given polarity.

3. The defibrillation signal as claimed in claim 1 or 2, characterized in that the spread of the known resistances of patients is divided into a plurality of ranges, and in that a distinct modulation law corresponds to each range.

4. The defibrillation signal as claimed in claim 1 or 2 or 3, characterized in that a phase of given polarity has a predetermined duration.

5. The defibrillation signal as claimed in the preceding claim, characterized in that the determined duration of each phase of given polarity is from 4 to 5 ms.

6. The defibrillation signal as claimed in any one of the preceding claims, characterized in that the defibrillation pulse is a biphasic defibrillation pulse with two phases of different given polarity to each other as a series of elementary pulses, of which the first of these elementary pulses is used for measuring the patient's transthoracic resistance with in view of modulating the following ones according to a modulation law in order to deliver a predetermined defibrillation energy while keeping substantially constant the total duration of the envelope of the series of elementary defibrillation pulses corresponding to at least one phase of given polarity.

7. The defibrillation signal as claimed in any one of the preceding claims, characterized in that the durations of the individual pulses of the second phase are controlled by using a single capacitor and biphasic defibrillation pulses, so as to dose the total electric charge or the total energy of the second phase so that the electric charge or the energy of the second phase has a desired proportion with respect to the electric charge or the energy of the first phase, without modifying the duration of this second phase.

8. The defibrillation signal as claimed in any one of the preceding claims 1 to 7, characterized in that the durations of the individual pauses of the second phase are controlled by using a single capacitor and biphasic defibrillation pulses, so as to dose the total electric charge or the total energy of the second phase so that the electric charge or the energy of the second phase has a desired proportion with respect to the electric charge or the energy of the first phase, without modifying the duration of this second phase.

9. A device for generating a defibrillation signal, in particular a signal as claimed in any one of claims 1 to 8, comprising defibrillation electrodes that can be applied to the patient, as well as means for delivering at least one defibrillation shock comprising at least one series of elementary pulses, characterized in that the device comprises
means for measuring the patient's resistance between electrodes applied to the patient
means for storing a plurality of truncation and modulation laws intended for modulating the widths of the elementary pulses and/or of the pauses between these pulses and/or the frequency of these pulses
and means for selecting a truncation law as a function of the measured resistance, in particular such that the energy applied to the patient is compensated and adjusted to a predetermined value.

10. The device for automatically compensating and adjusting the defibrillation or cardioversion energy as claimed in claim 9, characterized in that the high-voltage circuit (4) is composed of the following subassemblies: a high-voltage transformer and rectifier (A), means for storing the energy to be delivered to the patient (HVC), two semiconductor switches (B) and (C), an electromechanical switch (D), a module (E) for conditioning the voltage information, a module (F) for conditioning the current information and a module (G) for conditioning the control information.

11. The device for automatically compensating and adjusting the defibrillation or cardioversion energy as claimed in claims 9 and 10, characterized in that it includes two capacitors.

12. The device for automatically compensating and adjusting the defibrillation or cardioversion energy as claimed in claim 9, characterized in that it includes a single capacitor.

13. The device for automatically compensating and adjusting the defibrillation or cardioversion energy as claimed in the preceding claim, characterized in that it includes a bridge layout for discharging the single capacitor.

14. A method for generating a defibrillation or cardioversion signal that can be applied to a patient by electrodes connected to a defibrillator, characterized by the following steps:
Determination of the resistance between electrodes applied to a patient's body
Selection of a truncation law from a plurality of predetermined switching laws as a function of the measured resistance
Generation of a defibrillation wave consisting of at least one series of elementary pulses, of which the durations of these elementary pulses and/or the pauses between these pulses and/or the frequency of these pulses are modulated according to the truncation law.

15. A method for generating a defibrillation or cardioversion signal, in particular as claimed in claim 14, characterized in that the patient's transthoracic resistance is measured between electrodes applied to the patient by using the first elementary pulse of the defibrillation signal, and in that the following elementary pulses and the consecutive pauses are modulated according to a modulation law so as to form a signal that is dosed in energy according to a preselected or intended amount of energy, and to do so irrespective of the patient's measured transthoracic resistance and while keeping substantially constant the total duration of the envelope of the series of elementary defibrillation pulses corresponding to at least one phase of given polarity.

16. The method for generating a defibrillation or cardioversion signal as claimed in claim 14 or 15, characterized in that the defibrillation pulse is biphasic including two different phases of different given polarity.

17. The method for generating a defibrillation or cardioversion signal as claimed in claim 14 or 15 or 16, characterized in that the spread of the known resistances of patients is divided into a plurality of ranges, and in that a distinct modulation law corresponds to each range.

18. The method for generating a defibrillation or cardioversion signal as claimed in either one of claims 16 to 17, characterized in that a phase of given polarity has a predetermined duration.

19. The method for generating a defibrillation or cardioversion signal as claimed in claim 18, characterized in that the determined duration of each phase of given polarity is from 4 to 5 ms.

20. The method for generating a defibrillation or cardioversion signal as claimed in any one of claims 14 to 19, characterized in that the defibrillation pulse is a biphasic defibrillation pulse with two phases of different given polarity to each other as a series of elementary pulses, of which the first of these elementary pulses is used for measuring the patient's transthoracic resistance in view of modulating the following ones according to a modulation law in order to deliver a predetermined defibrillation energy while keeping substantially constant the total duration of the envelope of the series of elementary defibrillation pulses corresponding to at least one phase of given polarity.

21. The method for generating a defibrillation or cardioversion signal as claimed in any one of claims 14 to 20, characterized in that the durations of the individual pulses of the second phase are controlled by using a single capacitor and biphasic defibrillation pulses, so as to dose the total electric charge or the total energy of the second phase so that the electric charge or the energy of the second phase has a desired proportion with respect to the electric charge or the energy of the first phase, without modifying the duration of this second phase.

22. The method for generating a defibrillation or cardioversion signal as claimed in any one of claims 14 to 21, characterized in that the durations of the individual pauses of the second phase are controlled by using a single capacitor and biphasic defibrillation pulses, so as to dose the total electric charge or the total energy of the second phase so that the electric charge or the energy of the second phase has a desired proportion with respect to the electric charge or the energy of the first phase, without modifying the duration of this second phase.

23. The method for generating a defibrillation or cardioversion signal as claimed in any one of claims 16 to 21, characterized in that the duration of this second phase is selected so that its charge or energy content has a predetermined proportion with respect to the charge or energy content of the first phase.

24. The method for generating a defibrillation or cardioversion signal as claimed in any one of claims 14 to 22, characterized in that the measurement of the resistance and the selection of the truncation and modulation law are carried out during the first pulse, so that the cycle of this first pulse has a determined duty ratio with respect to at least one other duty ratio of the series of pulses.

25. The defibrillation signal as claimed in any one of claims 2 to 8, characterized in that the duration of the second phase is selected so that its charge or energy content has a predetermined proportion with respect to the charge or energy content of the first phase.

26. The defibrillation signal as claimed in any one of claims 2 to 8, characterized in that the second phase is not truncated into elementary pulses.

27. The defibrillation signal as claimed in the preceding claim, characterized in that the untruncated second phase has a duration such that its charge or energy content has a predetermined proportion with respect to the charge or energy content of the truncated first phase.

28. The defibrillation signal as claimed in any one of the preceding claims 2 to 8, characterized in that the resistance measurement is carried out during this first pulse, in order to control the duty ratio of the cycle containing the first pulse so that it has a predetermined value dependent on the measured resistance.

29. The defibrillation signal as claimed in claim 28, characterized in that the duty ratio of the cycle containing the first pulse is identical to the duty ratio of at least one of the consecutive cycles.
